(19) United States
(12) Patent Application Publication
(10) Pub. No.: US 2019/0374247 A1
(43) Pub. Date: Dec. 12, 2019

(54) Device to Assist in the Cannulation of Vascular or Other Anatomic Structures

(71) Applicant: Norman A. Paradis, Putney, VT (US)

(72) Inventor: Norman A. Paradis, Putney, VT (US)

(21) Appl. No.: 16/435,533

(22) Filed: Jun. 9, 2019

Related U.S. Application Data
(60) Provisional application No. 62/683,248, filed on Jun. 11, 2018.

(51) Int. Cl. A61B 17/34

(52) U.S. Cl. CPC A61B 17/3403; A61B 2017/00907

(57) ABSTRACT

Devices for improving vascular cannulation by increasing the probability of, and decreasing the time until, successful puncture of vascular and other structures. In its simplest form, a device composed of multiple connected needles in a linear or curvilinear array that is inserted perpendicular to the vessel such that one or more needles are likely to puncture the vessel. Alternatively, a gantry system incorporating a linear or curvilinear array of needle guides that allows sequential insertion of one or more needles in a linear pattern. The devices herein described may be used to successfully puncture any anatomic structure of interest.

### BACKGROUND OF THE INVENTION

Certain medical procedures and situations require cannulation of veins and arteries as quickly as possible. Examples would include emergency treatment for life-threatening shock, cardiac arrest and status epilepticus. Under such circumstances, medical therapies often must be initiated in the time-frame of seconds to minutes. Despite the need to initiate some therapies and procedures rapidly, clinical experience and the medical literature documents that this is not always achieved. (Lapostolle, Lininger) Delays in the initiation of emergent medical therapy may have multiple causes, but delays in achieving vascular cannulation are often disproportionately important. This is because, as the initial step in many therapeutic regimens, failure to rapidly achieve vascular access delays all subsequent steps.

Central to all modern methods of venous and arterial cannulation is the Seldinger guide wire technique.³ The Seldinger technique significantly increases the success of the catheter insertion step once the vessel has been punctured by the needle. It does not, however, assist in the necessary first step, successful puncture of the blood vessel.

For the purposes of this discussion, the basic steps in vascular cannulation include: 1) choice of appropriate vessel, 2) choice of needle (U.S. Pat. No. 2,409,979A), guide wire, dilator if needed, trocar/catheter, 3) sterilizing the insertion site 3) insertion of needle, 4) successful puncture of vessel, 5) insertion of guide wire (U.S. Pat. No. 3,547,103A, U.S. Pat. No. 3,521,620 A) through the needle and into the vessel, 6) removal of needle over guide wire, 7) insertion of the catheter over the guide wire, 8) removal of the guide wire, 10) connection of catheter hub to a fluid infusion system, 11) securing the catheter to the patient.

Of critical importance in successful vascular cannulation is accurate identification of vessel puncture. This event is associated with the operator stopping further insertion of the needle at that moment as further insertion would result in penetration through the back wall of the vessel. This would result in a loss of continuity between the needle lumen and the intravascular space.

Traditionally, successful vessel puncture is identified by the rapid presence of blood either in the needle hub or in a syringe attached to the needle hub. This event has been called blood “flashback.”

Recently, ultrasound imaging has been recommended to improve the safety and success rate of vascular catheter placement. Adjunctive use of ultrasound guidance may be associated with increasing success and decreasing injury. However, use of ultrasound may actually delay the time to vascular access secondary to the need to prepare and incorporate the transducer into the procedure while maintaining sterile technique.

Although certain steps in the placement of vascular catheters have seen improvements, the actual insertion of the needle into the vessel lumen is still dependent to a great extent on operator's skill. It may also be affected by the known biologic variation in anatomy. When time or resources do not allow use of ultrasound, there have been no significant improvements in the vessel puncture step of vascular access. The operator still holds the needle in their hand and inserts it at a position and along a vector that seems optimal based on their experience and knowledge of anatomy.

When the cannulation procedure is not successful on the first pass, the operator withdraws the needle, adjusts to an alternative location and vector and reinserts. This traditional method is problematical for a number of reasons:

1. Although the operator will attempt to visually remember the vector and depth of the previous failed attempt or attempts, human spatial memory is limited with respect to this type of data.

2. On additional attempts, the prior information from failed attempts is unlikely to be fully incorporated in the newest vector depths decision. It is possible that the operator will simply insert the needle in the direction and manner that is not significantly different from previous attempts with respect to the anatomy of the vessel in question.

3. Because the operator is holding the needle, it is unlikely that the vector and depth that is being attempted in the current pass will be stable. This limits the information that may be obtained from an unsuccessful insertion.

4. Because a human operator can only hold one needle, only a single insertion is attempted. There may be clinical circumstances in which insertion of more than one needle simultaneously may be optimal with respect to the probability of success and the risk-benefit ratio.

5. Because the operator is holding the needle, they may inadvertently change their orientation or depth after successfully puncturing the vessel. This may result in loss of successful puncture or unsuccessful insertion of the Seldinger guide wire.

6. Clinicians may be poor at judging the depths of their needle insertion, frequently inserting the needle either too shallow or too deep with respect to the intended vessel. In the latter case they may puncture the vessel and then proceed through the vessel and out the far wall, creating an injury without the benefit of successful cannulation.

7. The majority of humans have an essential tremor. During emergency procedures, this tremor is accentuated by endogenous release of catecholamines. Tremor during manual insertion of a needle acts as noise to the signal of correct orientation and depth.

8. Human practitioners holding a needle are subject to fatigue, which itself may exacerbate tremor, or cause loss of successful puncture before completion of guide wire passage.

9. The location and vector chosen for insertion of the needle may be compromised by the need for the operator to hold the needle during insertion. The operators position at the side of the patient, or limitations in their own musculoskeletal anatomy may impact the choice of location and vector.

10. Should the operator fail to puncture the intended vessel and withdraws the needle for another attempt, some of the anatomic and spatial information gleaned from the failed previous attempts is lost secondary to limitations in human spatial memory.

11. The tip of the needle may puncture the intended vessel, but the operator may fail to realize that this has occurred because entry of blood into the needle is not optimally visualized. This may cause the operator to continue insertion, exiting the far wall of the vessel, or may result in the operator withdrawing the needle and initiating an additional attempt.

12. The operator may successfully puncture the intended vessel and identify that this has occurred, but may lose the successful puncture through movement of the needle he is holding in his hand. This movement may result from normal human tremor or loss of attention when the operator turns to reach for the guide wire. Additionally, the needle may lose its position during the manipulation that may occur during insertion of the guide wire.

13. An additional limitation in current devices is inaccuracies in detection of blood “flashback” when the tip of the needle has punctured the vessel. While arterial vessels are generally at relatively high intravascular pressures, this is not the case for veins. The pressure within veins is generally either neutral or negative with respect to the environment. For this reason, successful puncture of a vein is not generally associated with reliable or rapid flashback of blood into the hub of the needle, where it can be detected by the operator. For this reason, venous cannulation is often attempted with a syringe attached to the needle hub and providing negative pressure because the operator is pulling on the syringe plunger. If, however, the operator does not apply optimal suction, early and reliable detection of flashback may be impaired or fail altogether.

Previous methods to achieve vascular access have been haphazard and not able to efficiently incorporate anatomic and spatial information from earlier failed attempts. If the vessel of interest is viewed as a target within the patient's tissues, then each failed insertion of the needle should be incorporated as a Bayesian prior in the spatial decision that is the next attempted insertion of the needle. The devices herein described supply a method to systematically optimize the manner in which the clinician assays the anatomic structures with which he is confronted. In particular these devices allow the retention of information gleaned from failed insertions of the needle and the application of this information systematically in subsequent attempts.

The devices and methods described herein are intended to address limitations in current techniques for achieving vessel puncture during vascular cannulation.

### FIELD OF THE INVENTION

The invention disclosed here relates in general to the field of medical devices used for achieving vascular cannulation, and more particularly, to a method for increasing the probability of successful vessel puncture during vascular cannulation and minimizing time to successful cannulation.

### DESCRIPTION OF THE RELATED ART

Previous methods and devices to improve the probability of vascular cannulation have focused on imaging techniques such as ultrasound (U.S. Pat. No. 6,132,379) with the intention of more accurately delineating the location of the needle. While such techniques may increase the probability of successfully puncturing a blood vessel with a single needle, they inherently create delay in vascular cannulation because of the time required for the imaging technique itself. Clinical practice has made evident that imaging-based techniques do not improve the rapidity of emergency vascular cannulation.

Survey of the medical and patent literature reveals no related system or method for improving the success of vascular cannulation by the methods and techniques described herein.

### BRIEF DESCRIPTION OF THE DRAWINGS

A further undrstanding of the nature and advantages of various embodiments may be realized by reference to the following figures.

FIG. 1—Depicts the multiprong comb needle device with hollow needles and their associated hubs in a linear array.

FIG. 2—Depicts the multi-pronged comb needle device with an adjustable retaining cross bar that may slide up and down the needles.

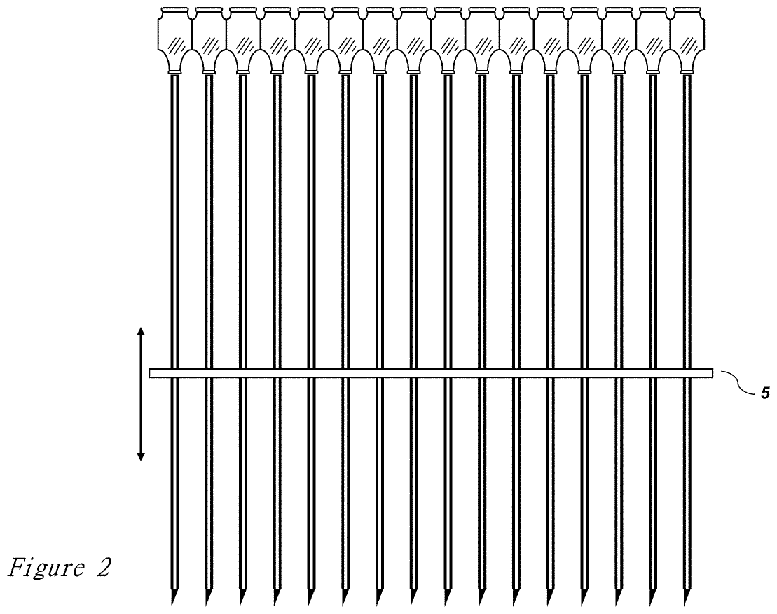

FIG. 3—Depicts a patient in the prone position with a comb needle being inserted into the femoral area of the groin.

FIG. 4—Depicts the gantry structural device for adjustable maintenance of the multi-pronged comb needle device orientation with respect to the patient. The comb needle device is loaded into the gantry.

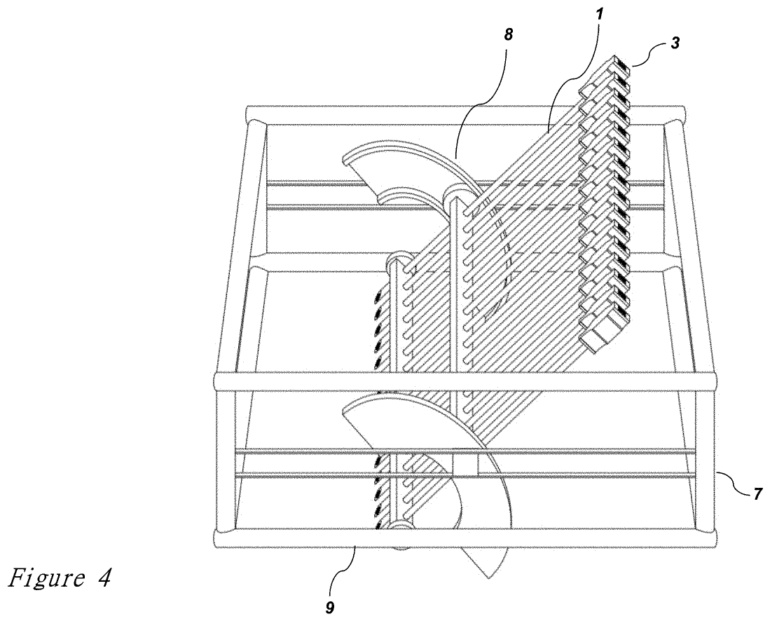

FIG. 5—Depicts the gantry structural device without the comb needle device. In this embodiment, the device is used to achieve a comb needle mechanism without an actual comb needle with hub-to-hub connections. Single hollow needles may be inserted sequentially one needle after another achieving a comb-needle functionality.

FIG. 6—Depicts the vacuum system attached to the comb-needle device, the comb-needle being mounted into the gantry structural device. The vacuum system is composed of pressure tubing attached to the hubs of each needle at one end and a vacuum manifold at the other.

FIG. 7—Depicts a single hollow needle with the transparent or translucent window to allow immediate detection of blood flashback. The internal surface of the needle and hub may be coated with a blood contrasting color to aid in immediate detection of blood flashback.

### SUMMARY OF THE INVENTION

In its simplest form, this method is a multi-pronged comb needle device 1. The device is composed of multiple needles individual needles 2, each with an individual hub 3, arranged in a linear or curvilinear manner and attached to the needles on either side at their hubs 3. Depending upon the clinical circumstances, the comb needle 1 may inserted perpendicular to the vascular structure of interest and with the center of the device located at the operator's best estimation of the vessel's location. Generally, the needles will be angled toward the desired J wire direction with the hubs 3 angled away. The comb needle is inserted until blood flashback is observed in one or more of the individual hubs 3. Standard Seldinger technique is then utilized. Under some circumstances, flashback will occur in more than one needle-hub 3 and the operator may choose the optimal needle-hub based on pattern and characteristics of blood flashback.

This multi-pronged comb needle will significantly increase the probability that successful puncture of the vessel will occur during the initial insertion. This should also be associated with a significant decrease in the time to successful vessel puncture and vascular cannulation. Because the vessel will likely be successfully punctured during initial insertion, use of the multi-pronged comb needle may be associated with less injury, as deeper structures will not be reached.

By way of additional expansion, the advantages of the multi-pronged comb needle may also be achieved by a gantry device that allows sequential insertion of one or more needles in a linear or curvilinear pattern. The gantry may have linear or curvilinear needle insertion guide hole bars that are adjustable with respect to patient angle. The needle insertion gantry would allow retention of the information provided by each failed insertion while minimizing the total number of needle insertions. This needle gantry might be adherent to the skin, providing additional advantages in terms of stabilization during subsequent steps in the cannulation procedure.

### DETAILED DESCRIPTION OF PREFERRED EMBODIMENTS

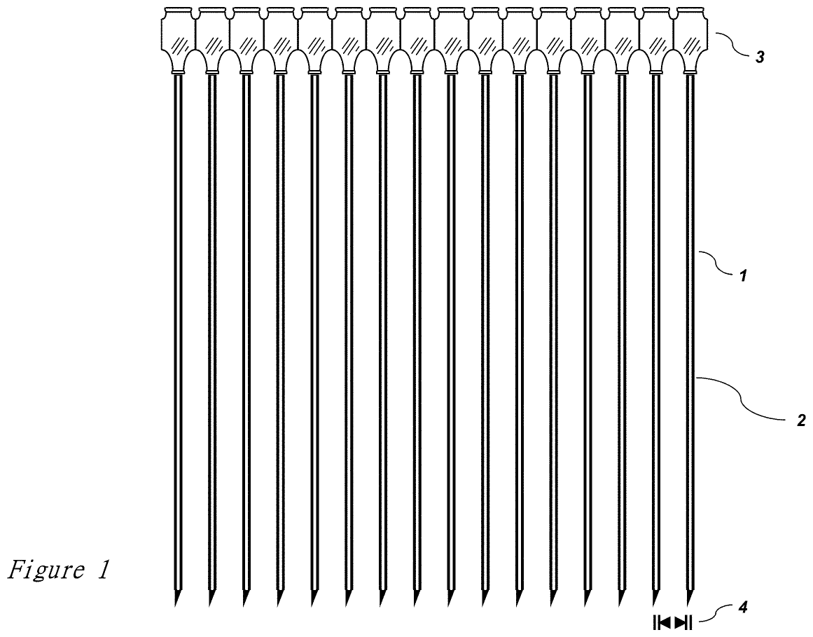

In its simplest form, this method is a multi-pronged comb needle device as in FIG. 1. The device is composed of multiple individual hollow needles 2, each with an individual hub 3, arranged in a linear or curvilinear pattern. Depending upon the clinical circumstances, the comb needle is inserted at a right angle to the vascular structure of interest and with the center of the device located at the operator's best estimation of the vessel's location. Generally, the needles 2 will be angled toward the desired J wire direction with the hubs 3 angled away. The comb needle is inserted until blood flashback is noted in one or more of the hubs. Standard Seldinger technique is then utilized. Under some circumstances, flashback will occur in more than one needle-hub and the operator may choose the optimal needle-hub based on pattern and characteristics of blood flashback.

By way of illustration but not limitation, additional refinements or improvements to the multi-pronged needle might include:

1. An adjustable retaining cross bar 5 which may be placed against the skin to maintain the needle angle and parallelism.

2. An adherent adjustable lockable retaining cross bar 5 which may be placed against the skin to maintain the relationship of the needle to the patient during follow-on portions of the cannulation procedure such as wire guide placement.

3. Variation in the inter-needle distance 4 selectable by the operator to match the clinical circumstances.

4. Variation in the number of needles, either by pre-selection or adjustable by operator. Said adjustment being made possible by breaking off unnecessary needles at pre-scored locations.

5. A multi-pronged needle in which individual needles could be broken off at each end until the total width is appropriate to the patient and clinical circumstances.

6. A pressure manifold mechanism 12 13 such that any number of needle hubs can be placed under partial vacuum 14 in a manner similar to the technique clinician's use with individual needle-syringe combinations. This will enhance blood flashback when the vessel of interest is punctured.

7. A multi-pronged stylet to prevent tissue from occluding needle lumens during insertion.

Hollow medical needles require a sharp bevel to puncture the skin and subcutaneous tissues. The orientation of the bevels may affect the likelihood of successful cannulation and Seldinger guide wire placement. In a particular embodiment of the comb needle, sharp bevels at the end of the hollow needles oriented at a 90-degree angle to the linear needle array. Hollow medical needles may become clogged with tissue when they are inserted into the patient. This will create a false negative with respect to puncture of the blood vessel. As blood is prevented from entering the hollow needle and needle hub. This false negative may be prevented by inserting a trocar into the hollow needle lumen so as to prevent tissue intrusion. In the case of the comb needle, this trocar can itself be multi-trocar in a manner that mates with the comb needle lumens.

The advantages of the multi-pronged comb needle 1 may also be achieved by a gantry structural device 7 10 that allows sequential insertion of one or more needles in a linear pattern. This would allow retention of the information provided by each failed insertion while minimizing the total number of needle insertions. This needle gantry might have adherent material on its patient side surface 9, providing additional advantages in terms of stabilization during subsequent steps in the cannulation procedure.

This gantry device and method is intended to hold a needle 2, needles, or needle guides 11 in a specific orientation to the patient and the vessel of interest, such that each insertion is optimized with respect to the information that is obtained and the probability of successful cannulation. The gantry mechanism 7 10 may be achieved by placing a linear or curvilinear array of needle guides in a 3-dimensional cube or rectangle that can be placed upon the patient. Alternatively, the needle guide array may be attached to a flexible arm that allows full 3-dimensional degrees of freedom and can be locked in place once its orientation to the patient and vessel of interest is deemed optimal by the clinician.

By way of additional expansion, the advantages of the multi-pronged comb needle may also be achieved by the gantry device without comb needle 10 that allows sequential insertion of one or more needles in a linear or curvilinear pattern. The gantry may have linear or curvilinear needle insertion guide hole bars 11 that are adjustable with respect to patient angle.

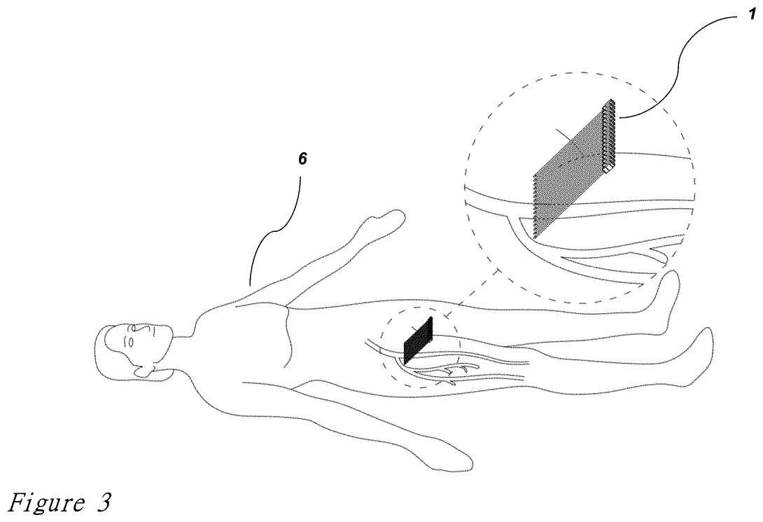

The first step in utilization of the gantry device would be to place the device in the appropriate location, such that the location and orientation of the needle, needles, or needle guides, appears optimal based on the operator's knowledge of anatomy. By way of example but not limitation, the femoral region of the groin might be selected FIG. 3 if it is the intention of the operator to cannulate either the femoral vein or artery.

The gantry device might be adherent to the patient, thus stabilizing its orientation during subsequent steps in the cannulation procedure. The gantry device might also be adjustable with respect to orientation even after it has become adherent to the patient.

Once the clinician has placed the gantry device on the patient, and adjusted and stabilized its orientation, he may then choose the needle or needles that will be initially inserted. This choice might be based on the urgency of cannulation and the operator's estimation of success. If relatively confident of success, or under conditions that are not emergent, the operator may insert only a single needle on the first pass. Under circumstances in which time is limited, or the operator is unsure of the anatomy, he may choose to insert multiple needles simultaneously, using the gantry device as if it were the simple comb needle configuration.

During the process of needle insertion, the operator controls the rate of insertion and the depth, and the components of the system maintain the needle location and vector in three dimensions. If the needle successfully punctures the intended vessel, the system maintains the position of the needle tip as the operator obtains and inserts the guide wire.

If the needle or needles fail to puncture the intended vessel, they can be left within the gantry system such that their vector and depth information can be incorporated fully as a true negative in choosing the next needle for insertion.

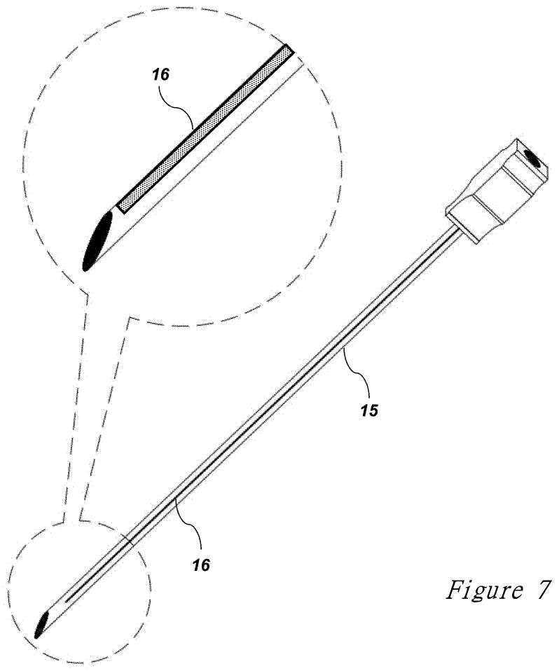

As an additional enhancement, the needles and their needle hubs may be designed in such a manner that detection of blood flashback is enhanced FIG. 7. This might be achieved by making some portions of the device, such as the needles of needle hubs, translucent or transparent 16. An additional enhancement would be to color the inside of the windowed needles or needle hubs a color that contrasts with blood.

In an alternative embodiment, the comb-needle device may incorporate hollow over-the-needle (U.S. Pat. No. 4,209,015A) catheters preloaded on the exterior of each hollow needle. The catheters would be inserted into vascular or other structures by advancement down the needles. The comb-needle is then removed and the catheter remains within the vessel. This cannulation technique is alternative to the Seldinger technique. It does not require insertion of a wire, as the catheter is directly inserted into the vein immediately after successful puncture. It is widely used in clinical medicine for placement of peripheral intravenous catheters.

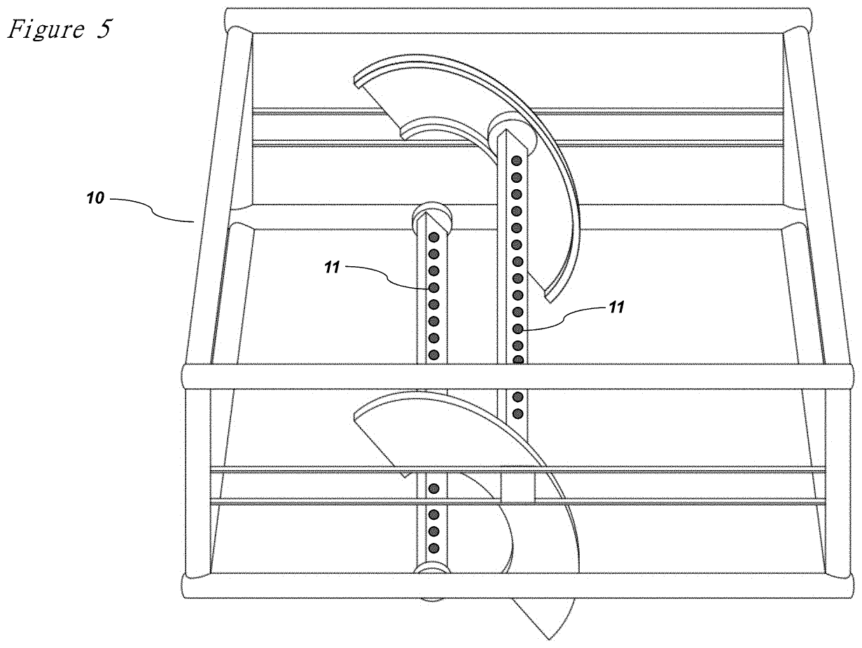

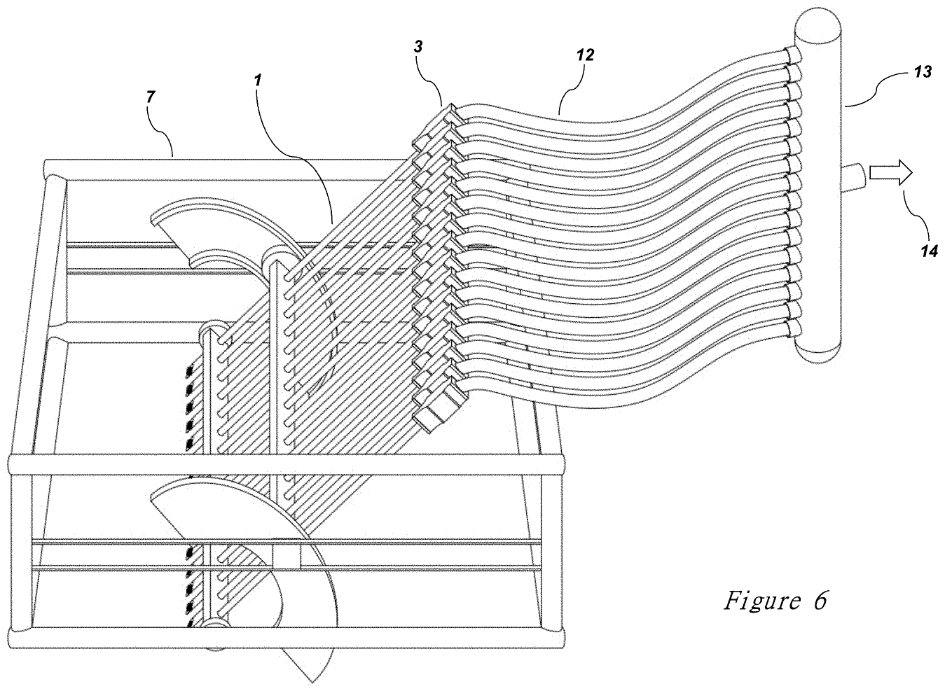

By way of additional expansion, the advantages of the multi-pronged comb needle may also be achieved by a gantry device that allows sequential insertion of one or more needles in a linear or curvilinear pattern FIG. 5 FIG. 6. The gantry may have linear or curvilinear needle insertion guide hole bars that are adjustable with respect to patient angle. The needle insertion gantry would allow retention of the information provided by each failed insertion while minimizing the total number of needle insertions. This needle gantry might be adherent to the skin, providing additional advantages in terms of stabilization during subsequent steps in the cannulation procedure.

The gantry embodiment of this invention might have a number of components or refinements:

An adherent stabilizing platform capable of holding one or more needles in a linear or curvilinear array.

An adherent stabilizing platform with antiseptic impregnation on its patient facing surface.

The needle holding gantry mechanism being capable of:
Maintaining a needle, needles, or needle guides in 3 dimensions.
Maintaining a needle, needles, or needle guides in a linear or curvilinear parallel pattern or any desired configuration.
Allowing simultaneous insertion of more than one needle. allowing addition of an automated needle hub vacuum system maintains needle orientation once puncture has occurred.
Allowing by addition or incorporation an imaging system based on ultrasound, regional oxygen saturation, or other indicator of vascular location.
A mirror or image enhancing device that allows the needle inserting operator to better visual the blood flash-back in the needle or needle-hub windows.
   The needles, themselves may have number of refinements:
1. are optimized to clinical application and vessel anatomy
2. are optimized for visual detection of flash back by incorporation of one or more of: windows, transparent or translucent hubs 15,
3. needles that are themselves transparent or translucent or that incorporate a linear window 16 and a white colored lumen so as to maximize identification of flashbacks once the vessel has been punctured.
4. optimized for Seldinger wire insertion.

The system being described is intended to hold a needle or needles in a specific orientation to the patient and the vessel of interest, such that each insertion is optimized with respect to the information that is obtained and the probability of successful cannulation. During the process of needle insertion, the operator controls the rate of insertion and the depth, the components of the system maintain the needle location and vector in three dimensions. If the needle successfully punctures the intended vessel, the system maintains the position of the needle tip as the operator obtains and inserts the guide wire. If the needle fails to puncture the intended vessel, it can be left within the system such that its vector and depth information can be incorporated fully into the next needle's insertion. One or multiple needles can be inserted simultaneously depending on the patient's anatomy and the urgency of the clinical circumstances.

The devices and methods herein described are intended principally for rapid and reliable cannulation of blood vessels. They can, however, also be used under any clinical circumstance in which access to an anatomic structure requires speed and reliability.

By way of example, but not limitation, a particularly comprehensive and flexible embodiment of the invention described herein might include:
1. a tubular gantry 7 10 with an adherent patient-side surface that might be placed over the vessel or hollow organ of interest.
2. The gantry containing at least two needle guide bars 11 with holes aligned to each other in a parallel linear pattern array.
3. A selection of hollow needles incorporating blood flash-back windows 16 and blood-contrasting inner surface coloring.
4. A mirror or imaging device visualizing the blood flashback windows.

An operator using this device would place the gantry over the vessel of interest after sterilizing the area. They might image the target vessels with techniques such as ultrasound. They would choose needle guide hole bars with inter-needle distances that reflect their estimate of the accuracy of the vessel location. They would then insert the first needle into the needle-guide holes at the lateral location of their best guess with respect to the vessel location watching for blood flashback in the flashback windows. If the first needle does not result in successful vessel puncture, the operator would then insert a second needle through the guide holes just next to the failed needle insertion. They would continue to repeat this pattern until successful puncture and cannulation.

It will be understood that many changes in the details, materials, steps and arrangements of elements, which have been herein described and illustrated in order to explain the nature of the invention, may be made by those skilled in the art without departing from the scope of the present invention.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents

### USEFULNESS OF THE DISCLOSED INVENTION

Once it is understood and appreciated that the inventions disclosed herein are for a method to increase the probability of successful vascular cannulation, the usefulness will be manifest to anyone with ordinary skill in the art.

Non-Obviousness

The non-obviousness of the invention herein disclosed is clear from the complete absence of its appreciation or discussion in the medical and patent literature. In particular, a number of the enhancements to the invention have not previously been disclosed. Additionally, many large commercial enterprises produce devices for vascular access and none of these companies have disclosed or developed devices, methods or systems such as disclosed herein.

### REFERENCE LIST

1. Shinar Z, Bellezzo J, Paradis N et al. Emergency department initiation of cardiopulmonary bypass: a case report and review of the literature. J Emerg Med 2012; 43(1):83-86.
2. Bellezzo J M, Shinar Z, Davis D P et al. Emergency physician-initiated extracorporeal cardiopulmonary resuscitation. Resuscitation 2012; 83(8):966-970.
3. Seldinger S I. Catheter replacement of the needle in percutaneous arteriography. A New Technique. Acta Radiol 1953; 39:368-376.
4. Lapostolle, F, Catineau, J, Garrigue, B, Monmarteau, V, Houssaye, T, Vecci, I & Adnet, F 2007, ‘Prospective evaluation of peripheral venous access difficulty in emergency care’, Intensive Care Medicine, vol. 33, no. 8, pp. 1452-1457.
5. Lininger, RA 2003, ‘Pediatric peripheral IV insertion success rates’, Pediatric Nursing, vol. 29, no. 5, pp. 351-354.

What is claimed is:

1) A device to increase the probability of, and decrease the time for, successful vascular or hollow organ cannulation, comprising:
multiple hollow sharp pointed needles;
each needle with a hub containing a central opening;
each needle attached in parallel to one or more adjacent needles such that the multiple needles are arrayed in a linear or curvilinear pattern.

2) The device of claim 1, also including sharp bevels at the end of the hollow needles oriented at a 90-degree angle to the linear needle array.

3) The device of claim 1, also including a linear stabilizing bar able to move freely up and down the needle shafts.

4) The device of claim 3, the linear stabilizing bar additionally having an adherent patient-side surface.

5) The device of claim 1, further comprising a multi-prong trocar that may be inserted into or removed from the needle lumens.

6) The device of claim 1, further comprising incorporation of transparent or translucent window areas within the needles or needle hubs.

7) The device of claim 6, further comprising a blood contrasting color on the inner surface of the hollow needle shafts or hubs.

8) The device of claim 6, further comprising incorporation of a needle windows image enhancement mechanism.

9) The device of claim 1, further comprising a sealed manifold capable of placed the needles and hubs under partial vacuum.

10) The device of claim 1, further comprising incorporation of imaging technology attached to the needle or hub array.

11) The device of claim 1, further comprising incorporation of electrical conducting paths specific to individual needles.

12) The device of claim 10, further comprising electrical circuitry capable of measuring a plurality of the impedance, resistance, or current flow specific to each needle in the array.

13) A device to increase the probability and decrease the time for successful vascular or hollow organ cannulation, comprising:
a gantry structure holding in three dimensions the location and orientation of multiple parallel independent needles;
and allowing selective insertion of each needle independent of the others.

14) The device of claim 13, further comprising substitution of needle guide holes for the needles.

15) The device of claim 13, further comprising a mechanism to adjust and hold the needle array at a specific orientation to the patient.

16) The device of claim 13, further comprising incorporation of a needle windows image enhancement mechanism into the gantry.

17) The device of claim 13 also including an adherent patient-side surface.

18) The device of claim 1, further comprising incorporation of over-the-needle catheters preloaded on the exterior of each hollow needle.
